(19) United States
(12) Patent Application Publication
(10) Pub. No.: US 2009/0246751 A1
(43) Pub. Date: Oct. 1, 2009

(54) METHOD FOR THE DETECTION OF VIRUS INFECTED CELLS

(75) Inventors: Peter Ruhdal Jensen, Gentofte (DK); Ole Michelsen, Tisvildeleje (DK)

Correspondence Address: FOLEY AND LARDNER LLP;SUITE 500, 3000 K STREET NW, WASHINGTON, DC, 20007, US

(73) Assignee: DANMARKS TEKNISKE UNIVERSITET

(21) Appl. No.: 12/160,876

(22) PCT Filed: Jan. 16, 2007
(86) PCT No.: PCT/DK2007/050001

§ 371 Date: Dec. 22, 2008

Related U.S. Application Data
(60) Provisional application No. 60/759,071, filed on Jan. 17, 2006.

(51) Int. Cl. C12Q 1/70

(52) U.S. Cl. 435/5

(57) ABSTRACT

The present invention relates to a method for determining one or more stressed cell(s) in a medium. The method comprises the steps of (i) providing a sample from the medium, and (ii) determining a change in the sample relative to a normal sample of non-stressed cell(s).

### FIELD OF THE INVENTION

The present invention relates to a method for the detection of stressed cells in a sample. In particular, the present invention relates to a method for the detection of general and unspecific virus infected cells based on determining a change in the sample relative to a normal sample of non-stressed, in particular non-virus infected cell(s) using flow cytometric technology.

### TECHNICAL BACKGROUND AND PRIOR ART

A virus is a noncellular agent that is capable of reproducing only in an appropriate host cell. The virus cell comprises a DNA or a RNA sequence which is surrounded by a capsid coat and when infecting a cell the virus takes over the control of the cell and utilises the capacity of the cell.

The generally acknowledged action performed by viruses involves 5 steps:
(i) Attachment of the virus to specific sites on the surface of the host cell,
(ii) Penetration of the cell membrane and the “tail” of the virus contacts and pushes a hole through the cell membrane and injects its DNA through the cell membrane and into the cytoplasm of the host cell.
(iii) Replication begins once the virus DNA gets inside the cell and takes over the metabolic machinery of the cell. By using the host cells ribosomes, its energy and many of its enzymes, the virus replicates its own macromolecules. Virus genes contain all the information necessary to produce new viruses.
(iv) Assembly of the newly synthesised viral components into new viruses within the host cell.
(v) Release of the newly synthesised viruses from the host cell by the secretion of an enzyme that degrades the cell membrane and releases the viruses. An entire lytic cycle, from attachment to release, takes approximately 30 minutes and about 100 new viruses are released ready to infect other cells.

The infection of cell cultures by viruses is generally a problem in industries, such as in the dairy industry, where the virus attack in the fermentation process are mostly observed very late in the process by a decrease in the acidification at which point it is too late to intervene. The actions to overcome the attack observed late are limited or require hazardous treatment or financial heavy involvement to defeat the virus attack.

One way of detecting viruses is by subjecting the DNA sequence to polymerase chain reaction (PCR) in order to amplify the signal from the virus infected cells and thereby be able to detect the infection as early as possible. One problem with this type of detection of the virus infected cells is the time required for the PCR-reaction before a result is ready and a decision on whether the culture is infected or not can be taken.

Additionally, viruses can be specifically detected based on the DNA sequence contained in the virus, in which case the DNA sequence is labelled with a specific binding pigment which subsequently is detected in a flow cytometer to give an indication of the amount or numbers of viruses present in the sample. Unfortunately, viruses contain different DNA sequences and therefore, different methods are needed to determine different types of viruses. The result is that the person skilled in the art needs to have a thoroughly understanding of the viruses likely to be present in the sample and then direct the analyses for these particular viruses.

Several publications are provided (see Dominique et al. (1999); Haidinger (2003); Sheehan et al. (2005) and Ananta E. (2005)) which uses flow cytometry as an effective tool for discriminating between cells which are virus infected and cells which are not virus infected. However, all these publications measures whether the cells are dead or alive. The publications do not describe anything about differentiating healthy live cells from stressed and/or infected live cells.

Thus, it is desired by the industry to be able to detect stressed cells, such as virus infected cells in a quick, easy and reproducible manner and without being limited to measuring viruses which are known in advance to the person skilled in the art. Thereby, a method for non-specific determination of stressed cells, such as virus infected cells, is provided.

### SUMMARY OF THE INVENTION

Accordingly, the present invention provides in a first aspect a method for determining one or more stressed cell(s) in a medium, said method comprises the steps of:
(i) providing a sample from the medium, and
(ii) determining a change in the sample relative to a normal sample of non-stressed cell(s).

In a further aspect of the present invention an automatic or semiautomatic system is provided. The system comprises the following interconnecting means:
(a) means for collecting a sample from a medium,
(b) means for observing a change in the sample relative to a normal sample.
   wherein the change in the sample is based on whether a cell present in the sample is stressed or not.

### DETAILED DISCLOSURE OF THE INVENTION

The inventors of the present invention has surprisingly found that when cells are stressed they are significantly differentiated from normal cells (non-stressed cells) when subjected to e.g. a flowcytometric technology. Normally, stressed cells are detected by analysing the factors causing the cells to be stressed, whereas the present invention is related to the change in the behaviour of the stressed cells. This new approach of analysing samples makes it possible to detect the stressed cells at an earlier stage. For instance in the dairy industry the acidification of milk performed by lactic acid bacteria may be contaminated by virus infection of the lactic acid bacterium, which thereby stresses the cells.

Conventionally, such infected cells were not detected until the virus infection is distributed to such an extent that it is too late to act and the medium may be discarded. The present invention provides a method and a system for detection of stressed cells (such as virus infected cells) in time to act e.g. by the addition of an additional starter culture or any other appropriate action.

To be able to act in time the present invention relates to a method for determining one or more stressed cell(s) in a medium, said method comprises the steps of: (i) providing a sample from the medium, and (ii) determining a change in the sample relative to a normal sample of non-stressed cell(s).

In the present context the term “stressed cell(s)” relates to live cells that have been exposed to one or more conditions causing the cell to act different from normal cell(s) and samples. The term “stressed cell(s)” does not relate to dead cell(s). The differences in the stressed cell(s) make it possible to determine the change in the sample relative to the normal sample. In an embodiment of the present invention the cell(s) may be stressed by means selected from the group consisting of deviating temperature, salt concentration, virus infection, starving the cells, such as limiting the nitrogen source and limiting the carbon source.

In yet an embodiment of the present invention the change may be determined by e.g. subjecting the sample to flow cytometry technology, such as forward light scatter, sideward light scatter, fluorescent signal and any combination thereof, to obtain one or more flowcytometric histograms and/or one or more cytograms of said sample. Furthermore, the change may be determined by subjecting the sample to at least one additional analysis selected from the group consisting of determination of the DNA content, determination of the electrical potential over the cell membrane, internal cytoplasmic pH and specific host proteins induced by infection (e.g. fluorescence labelled antibodies).

In the present context the term “flow cytometry technology” relates to the measurement of physical and/or chemical characteristics of cells, or, by extension, of other biological particles. Flow cytometry is a process in which such measurements are made while the cells or particles pass, preferably in single file, through the measuring apparatus in a fluid stream. As an addition, flow sorting extends flow cytometry by using electrical or mechanical means to divert and collect cells with one or more measured characteristics falling within a range or ranges of values set by the user.

The result obtained from the flow cytometry technology may be presented in the form of a flow cytometric histogram and/or one or more cytograms of said sample in respect of the selected characteristics. Generally, the flowcytometric histogram is a 2D graphic plot representing number of cells in one direction versus signal in the other direction. The cytogram is a multi-D graphic plot representing signals from the various parameters detected together with number of cells in each point.

When a cell is being stressed, it may be apparent from the behaviour of the cell and from different characteristics of the cell. In the present context the term “at least one additional analysis” relates to test methods to be performed in order to increase the determination of stressed cells. In an embodiment of the present invention the at least one additional analysis may be performed by determining the DNA content of the cell, determining the electrical potential over the cell membrane, measuring the internal cytoplasmic pH or if specific host proteins induced by infection (e.g. fluorescence labelled antibodies).

In order to determine a change in the bacterium it is necessary to determine or define a normal sample. In the present context the term “normal sample” relates to a signal from e.g. the flow cytometer arranged in a multi-dimensional space together with the number of cells having the specified values (normal values), which will then become the normal situation (normal picture) from which any significant and unexpected change. Such a significant and unexpected change may trigger a signal to alert an operator. Signals may be signals for forward light scatter; sideward light scatter; DNA content; membrane potentials; internal cytoplasmic pH; specific host proteins induced by the infection (fluorescence labelled antibodies).

In an embodiment of the present invention the determination of the stressed cells is performed as an at-line or on-line determination, preferably as an on-line determination.

In addition to providing a method for determining one or more stressed cells in a medium, the inventors of the present invention have also provided an automatic or semiautomatic system comprising the following interconnecting means: (a) means for collecting a sample from a medium, (b) means for observing a change in the sample relative to a normal sample, wherein the change in the sample is based on whether a cell present in the sample is stressed or not.

In addition to the above mentioned means the system may further comprise means for storing data, including data obtained from the flow cytometer, preferably in form of the readable signal.

In an embodiment of the present invention, the means for collecting a sample is being controlled by the means for storing data for the surveillance of stress development in said medium such that the means for collecting a sample is only activated at pre-selected points in time or at pre-selected time intervals.

It is a feature of the present invention that samples are provided and analysed in a dynamic and intelligent mode, i.e. that the means for collecting a sample is only activated at a given point in time during the growth phase of the cells. This may be achieved by providing a computer system for storing data including data for the specific characteristics of normal cells and stressed cells. Such specific characteristics may be determined by e.g. flow cytometry technology, such as forward light scatter, sideward light scatter, fluorescent signal and any combination hereof. Furthermore, the specific characteristics may be determined by the DNA content, the determination of the electrical potential over the cell membrane, the internal cytoplasmic pH, change in the production of organic acids or by specific host proteins induced by infection (e.g. fluorescence labelled antibodies).

Furthermore, the determination of whether a sample shall be analysed or not may be controlled by the computer having data stored for the specific characteristics of normal cells and stressed cells such that the means for observing a change is only activated at selected points in time or at selected time intervals. In this connection, one interesting feature is that the computer having data stored for the specific characteristics of normal cells and stressed cells is continuously updated with new data, so that the frequency of samples to be analysed is dynamic and based on a constantly updated set of data for the particular population member.

In another embodiment of the present invention, the means for collecting a sample and/or the means for observing a change in a sample is/are continuously activated. This means that analysis is performed at substantially fixed time intervals between each sample whereby samples are provided and analysed without stopping or without interruption of the substantially fixed time intervals between each sample to be analysed.

In the system according to the present invention, the means for observing the change relates to or involves flow cytometry technology, such as forward light scatter, sideward light scatter, to obtain flowcytometric histograms and/or cytograms of said sample.

In an embodiment of the present invention, a sample is provided and measured directly by pumping a small fraction of the medium through a staining procedure into e.g. a flow cytometer. In an embodiment of the present invention, the pumping of the small fraction of the medium may be performed continuously.

To measure the change in a sample, such a sample must be provided by a subset of the medium which is suspected of containing stressed cells and the sample must be in the form of a liquid, a solution, a suspension or any other kind of aqueous phase or fluidised phase, preferably capable of being subjected to flow cytometry technology. In the present context the term “medium” relates to the source which is suspected of containing stressed cells. In an embodiment of the present invention the medium may be a meat product, a vegetable fermentation medium, a product from the dairy industry, such as milk or a medium from the cheese production, a product from the brewery industry, a product from the pharmaceutical industry, or any other kind of fermentation industry. In another embodiment of the present invention, the medium may be a small scale fermentation or a large scale fermentation.

In some cases, the sample obtained from the medium needs to be clarified, at least to some extent, before the sample is being applied to the flow cytometer. Methods for performing such clarifications steps are dependent on the medium and are well known for the person skilled in the art. Preferably, such clarification step has no influence as such on the determination of the stressed cells.

In the present context of the present invention the term “cell(s)” is selected from the group consisting of prokaryotic cell and eukaryotic cell. Preferably the cell is a prokaryotic cell and preferably the prokaryotic cell is a bacterium.

In the scope of the present invention the bacterium relates to any kind of industrially used bacterium. Preferably, the bacteria may be selected from the group consisting of a Gram positive bacterium and a Gram negative bacterium.

In an embodiment of the present invention the Gram negative bacteria is a lactic acid bacterium, such as Lactococcus species, Lactobacillus species, Streptococcus species, Oenococcus species, Leuconostoc species and Pediococcus species, Enterococcus species, including strains of the species Lactococcus lactis and Streptococcus thermophilus

In another embodiment of the present invention the eukaryotic cell is selected from the group consisting of an animal cell, a fungi cell and a yeast cell.

As stated above, the conventionally used methods have such as poor sensitivity that, when the stress is detected it is to often too late to act and the medium may be discarded. However, the method and the system described herein provide a sensitivity which is high and provides time to act on the stressed cells, whereby the medium in most cases does not need to be discarded. In the present context, the term “sensitivity” relates to an index of the ability of the detection procedure described in this method or for this system to make quantitative determinations at low levels. In an embodiment of the present invention, the system or the method has a sensitivity for measuring the number of stressed cells relative to the total number of cells which is 5% or less, such as 2% or less, e.g. 1% or less, such as 0.5 or less, e.g. 0.1 or less, such as 0.05% or less, e.g. 0.01% or less, such as 0.005 or less, e.g. 0.001 or less.

When determining the change in the stressed cells relative to the normal cells, the change may be seen in a graphic plot such as in a flowcytometric histogram or a cytogram. In this graphic plot, the normal cells will be found in approximately the same area surrounding a peak area, which may be defined as 100% of normal cells. The normal distribution of the cells may be distributed around the peak area by 5%. Thus, the normal cells may normally be found in the range of 95% to 105% of the peak area. It is to be understood that certain deviations from this range may be found.

In an embodiment of the present invention, the stressed cells may be found in a flowcytometric histogram or a cytogram in the range of from 0% to 95% relative to the normal cells, such as 0-70%, e.g. 0-60%, such as 0-50%, e.g. 10-40%, such as 10-30%, e.g. 10-20%. In a further embodiment of the present invention the stressed cells may be found in a flowcytometric histogram or a cytogram in the range of from 105% and above relative to the normal cells, such as 110% and above, e.g. 120% and above, such as 130% and above, e.g. 140% and above, such as 150% and above, e.g. 200% and above.

In a further embodiment of the present invention the stressed cells peaks in a flowcytometric histogram or a cytogram in the range of from 0% to 90% relative to the normal cells, such as 0-70%, e.g. 0-60%, such as 0-50%, e.g. 10-40%, such as 10-30%, e.g. 10-20%. In yet a further embodiment of the present invention the stressed cells peaks in a flowcytometric histogram or a cytogram in the range of from 105% and above relative to the normal cells, such as 110% and above, e.g. 120% and above, such as 130% and above, e.g. 140% and above, such as 150% and above, e.g. 200% and above.

It is also a surprising effect of the present invention that the determination of the stressed cell is independent of the influence on the specific characteristic causing the cells to be stressed. In particular, the determination of one or more virus infected cell(s) is/are independent of the type of virus and/or whether one or more type(s) of virus is/are infecting.

The most pronounced change in the cells may be observed when the virus is a bacteriophage, such as a bacteriophage that infects Gram positive bacteria, such as a bacteriophage that infect lactic acid bacteria, such as a bacteriophage that infects lactococcus species, such as lactococcus phages selected from the group consisting of type 936, type P335 and type C2.

In an embodiment of the present invention, a computer may be used for observing the change in the sample caused by the virus infected cell(s). Preferably, the computer may be provided with a suitable computer program for detecting the change. The result may subsequently be shown in such a manner that the stressed cells are clearly and significantly differentiated from the normal cells.

The invention will now be further illustrated by the following non-limiting drawings and examples.

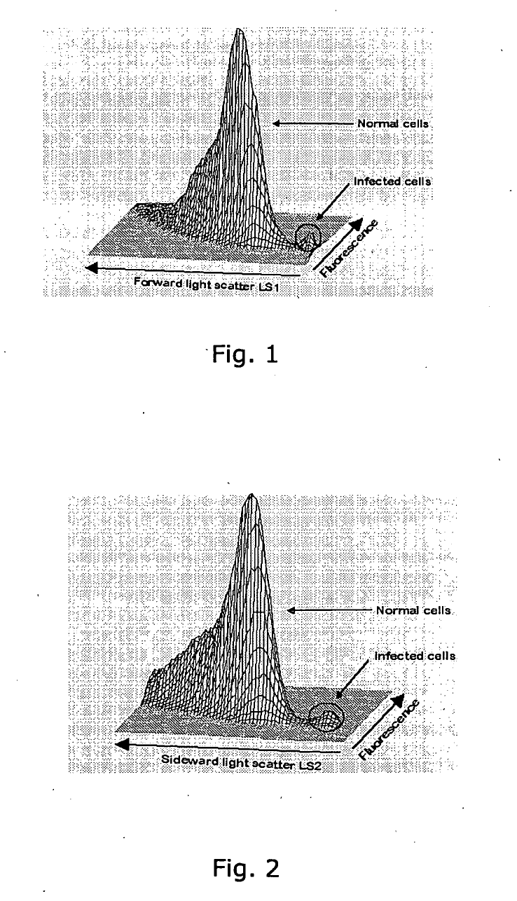

FIG. 1 shows a flow cytometric cytogram (forward light scatter versus DNA fluorescence) for a sample withdrawn at 80 minutes after infection. The infected cells can be clearly identified separated from the non-infected cells.

FIG. 2 shows the same sample as in FIG. 1. The cytpgram has been changed to sideward light scatter versus DNA fluorescence. It is clearly seen that in this cytogram the infected cells are more separated from the axis than in FIG. 1.

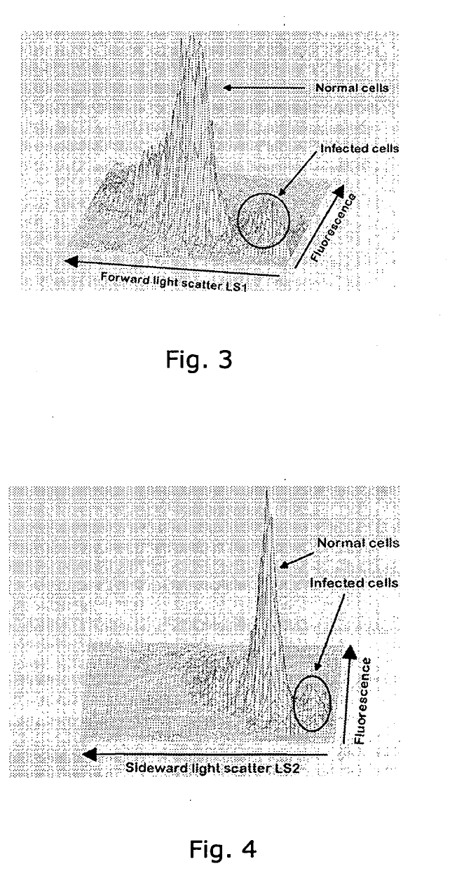

FIG. 3 shows that the detection of the infected cells can be done in milk using forward light scatter. Separation from the axis is better than in the previous experiment. (see FIG. 1)

FIG. 4 shows sideward light scatter, of infected cells in milk, giving a clear separation of infected cells from the normal uninfected cells.

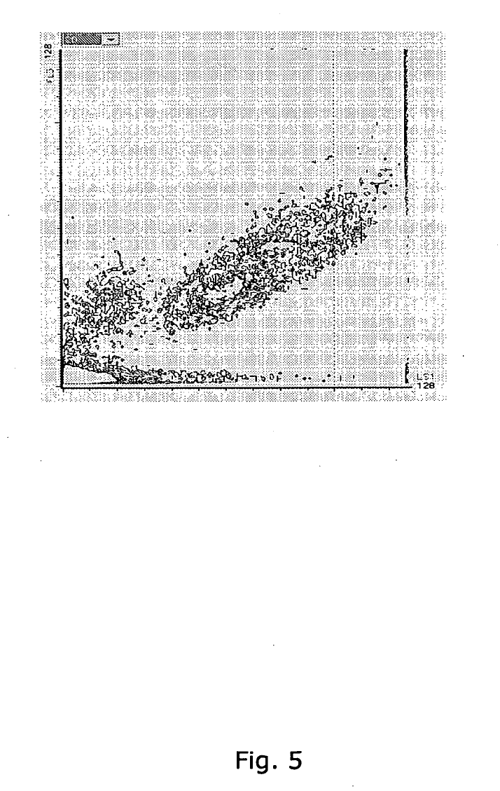

FIG. 5 shows the data in FIG. 3 seen as a line plot. The non fluorescence material in the lower left corner shows small particles from the medium.

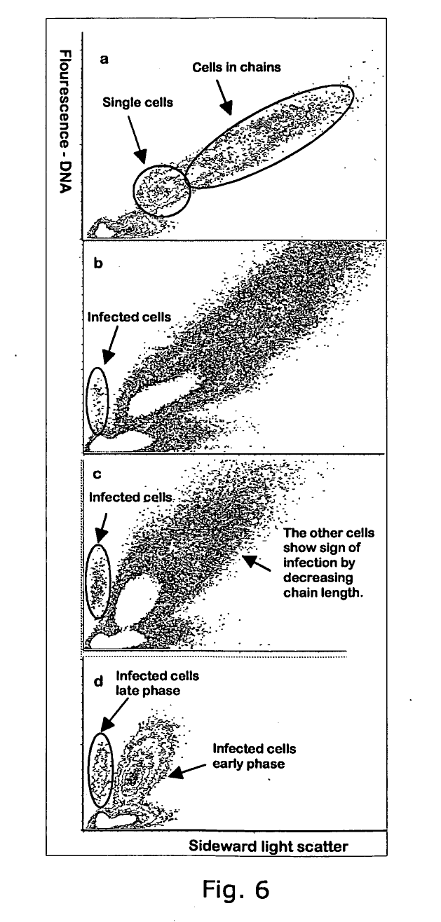

FIG. 6 shows the scale for cell number which are different in the four panel. In panel a) and d) the maximum line goes through points with 200 cells, while in panel b) and c) the maximum line goes through points with 20 cells.

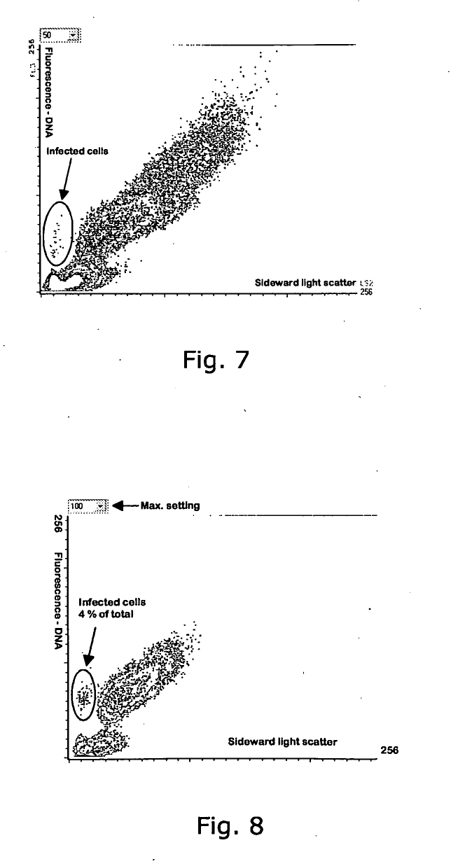

FIG. 7 shows the earliest identification of the infected cells, when infected with the phage wj9B (1.4% of total).

FIG. 8 shows the earliest identification of infected cells, when infected with the phage wj16B (4% of total).

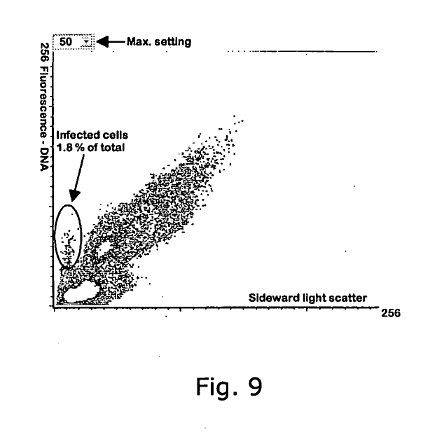

FIG. 9 shows the earliest identification of infected cells, when infected with the phage wj30B (1.8% of total).

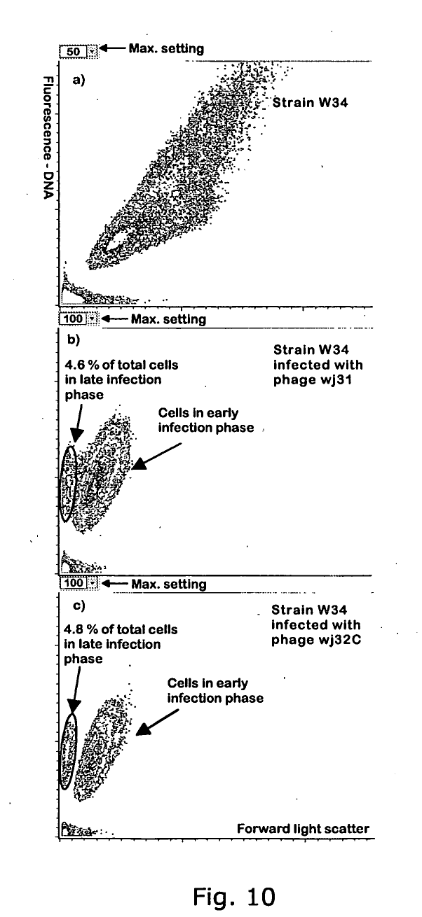

FIG. 10 show forward light scatter against fluorescence—DNA. Panel a) shows a flow cytogram of the uninfected host Lactococcus lactis W34. Panel b) shows Lactococcus lactis W34 infected with phage wj31. Panel c) show Lactococcus lactis W34 infected with wj32C.

### EXAMPLES

### Example 1

The purpose of this experiment was to find the earliest sign of an infection in a culture of Lactococcus lactis subsp. cremoris Mg1363.

M17 2% glucose medium was inoculated with the bacterium. When growth was established the culture was infected with 3000 Lactococ phage C2 per ml. Thereafter growth was followed and samples for flow cytometry were withdrawn every 10 minutes.

The samples were additionally stained for DNA and then subjected to flow cytometry in a Bryte instrument (3-5).

### Photomultiplier Settings: LS1 500V, LS2 590V, F13 500V

### Gain: LS1 2, LS2 20, FL3 5.

The sample withdrawn at 80 minutes after infection was the first sample in which infected cells were detected using Forward light scatter (LS1) vs. fluorescence (FIG. 1) and Sideward light scatter (LS2) vs. fluorescence (FIG. 2).

The result shows that the infected cells (1.5% of total) can be seen clearly separated from the non-infected cells and that the infected cells measures by sideward light scatter are more separated from the axis than the infected cells measures by forward light scatter.

### Example 2

In this experiment the aim was to show if the detection could be done in milk.

MG1363 was inoculated in a mixture of skimmed milk and M17 with lactose as carbon source. M17 was used because MG1363 does not harbor the plasmid with protease that dairy strains usually do. A sample was withdrawn before infection and then one late in infection. The two samples, 200 μl of the sample from before infection and 20 μl of the sample late in infection, were mixed.

The mixture was cleared with polyphosphate (1 M final concentration) then stained for DNA and run through the Bryte flow cytometer.

### Setting Used:

### Photomultipliers: LS1 500 V, LS2 575 V, FL3 525 V

### Gains: LS1 5, LS2 10, FL3 5

The experiment shows that the detection of the infected cells can be done in milk (FIG. 3 and FIG. 4) and it can be seen that the DNA staining serves to separate the cells from the nonliving particles present in the medium (FIG. 5)

### Example 3

This experiment is the first of a series where a collection of phages—of the P335 type—isolated from a cheddar plant were tested. These phages have a common host the Lactococcus lactis strain W34 that was grown in M17 with 20% glucose. The culture was infected with phage jw1.

Samples were withdrawn at different times during the infection, stained and run through the flow cytometer with the settings.

### Photomultipliers: LS1 475 V, LS2 600 V, FL3 525 V

### Gains: LS1 10, LS2 10, FL3 10

In FIG. 6 four studies can be seen. The scales for cell number are different in the four panels. In panel a) and d) the maximum line goes through points with 200 cells, while in panel b) and c) the maximum line goes through points with 20 cells. The changes have been done in order to visualize the small fraction of infected cells in the area to the left for the uninfected cells.

Panel a) shows a normal picture of this W34 strain. The cells lie in the diagonal of the picture because the cells grow in chains.

Panel b) shows cells from an infected culture just as the first sign of infected cells can be seen (0.6% of total).

Panel c) shows cells from the culture later in the infection. The infected cells are clearly seen. The other cells show sign of early phase infection (the cells change toward growing as single cells).

Panel d) shows cells late in the infection. A small fraction of cells in the late infection phase the other clearly as single cells (sign of an early infection phase).

This experiment demonstrates that the first sign of infected cells is the small fraction of infected cells as seen in panel b); but later in the infection a change from chains to single cells can be seen. The fraction of infected cells with less light scatter stays low because these cells represent cells late in the infection—just prior to lyses.

### Example 4

This experiment was performed in the same manner as in example 3 with the same settings but with phage wj9B (1).

FIG. 7 shows that the earliest identification of infected cells, when infected with phage wj9B, infected cells was 1.4% of total. The major parts of the other cells were still unaffected by the infection.

### Example 5

This experiment was performed in the same manner as in example 3 but with phage wj16B instead.

FIG. 8 shows that the earliest identification of infected cells, when infected with phage wj16B, was approximately 4% of the total cells.

### Example 6

This experiment was performed in the same manner as in example 3 but with phage wj30B instead.

FIG. 9 shows that the earliest identification of infected cells, when infected with phage wj30B, was approximately 1.8% of the total cells.

### Example 7.

This experiment explores the observation that the first sign of infection actually may be the change from growth in chains toward single cell growth. The experiments were performed as in example 3 but with different phages and different settings.

### Photomultipliers: LS1 500 V, LS2 650 V, FL3 530 V

### Gains: LS1 5, LS2 5, FL3 10

The cytogram in FIG. 10 show forward light scatter against fluorescence—DNA. Panel a) shows a flow cytogram of the uninfected host Lactococcus lactis W34. Panel b) shows Lactococcus lactis W34 infected with phage wj31. Panel c) show Lactococcus lactis W34 infected with wj32C.

The samples were withdrawn just prior to lyses of the cultures. It can be seen that only a fraction of the cells were in the areas where the earliest signs of infections were seen. The major parts of the cells were found in single cells and were present in an area of the cytograms with a lower light scatter than the uninfected host. These samples have been withdrawn later in the infection than the sample in panel c) in FIG. 6. From this experiment it can be concluded that the cells with very low light scatter seen early in infection only represent a fraction of the infected cells late in the infection just prior to lyses. Earliest sign of infection is then the change toward single cell growth and a small drop in light scatter. This change in behaviour could be found with a computer analysis of the changes in the distribution of the cells in multidimensional cytograms.

1. A method for determining one or more virus infected cell(s) in a medium, said method comprises the steps of:
(i) providing a sample from the medium, and (ii) determining a change in the sample relative to a normal sample of non-virus infected cell(s), by subjecting the sample to flow cytometry technology, such as forward light scatter, sideward light scatter, fluorescent signal and any combination hereof, to obtain one or more flowcytometric histogram and/or one or more cytogram of said sample,
wherein the cell is a prokaryotic cell.

2. A method according to claim 1, wherein the sample is subjected to at least one additional analysis selected from the group consisting of determination of the DNA content, determination of the electrical potential over the cell membrane, internal cytoplasmic pH and specific host proteins induced by infection.

3. A method according to claim 1, wherein the determination is performed as an at-line or on-line determination, preferably as an on-line determination.

4. A method according to claim 1, wherein a sample is provided and measured directly by pumping a small fraction of the medium through a staining procedure into a flow cytometer.

5. A method according to claim 1, wherein the sample is continuously provided and analysed by pumping a small fraction of the medium through a staining procedure into a flow cytometer.

6. A method according to claim 1, wherein the medium is a small scale fermentation or a large scale fermentation.

7. A method according to claim 1, wherein the medium is obtained from a meat product, a vegetable fermentation, the dairy industry, such as milk, the brewery industry, the pharmaceutical industry, or any other kind of fermentation industry.

8. A method according to claim 1, wherein the prokaryotic cells is a bacterium.

9. A method according to claim 8, wherein the bacteria is selected from the group consisting of a Gram positive bacterium and a Gram negative bacterium.

10. A method according to claim 9, wherein the Gram negative bacteria is a lactic acid bacterium, such as Lactococcus strains and Lactobacillus strains.

11. A method according to claim 1, wherein the method has a sensitivity for measuring the number of stressed cells relative to the total number of cells which is 5% or less, such as 2% or less, e.g. 1% or less, such as 0.5 or less, e.g. 0.1 or less, such as 0.05% or less, e.g. 0.01% or less, such as 0.005 or less, e.g. 0.001 or less.

12. A method according to claim 1, wherein the stressed cells is found in a flowcytometric histogram or a cytogram in the range of from 0% to 95% relative to the normal cells, such as 0-70%, e.g. 0-60, such as 0-50%, e.g. 10-40, such as 10-30%, e.g. 10-20.

13. A method according to claim 1, wherein the stressed cells is found in a flowcytometric histogram or a cytogram in the range of from 105% and above relative to the normal cells, such as 110% and above, e.g. 120% and above, such as 130% and above, e.g. 140% and above, such as 150% and above, e.g. 200% and above.

14. A method according to claim 1, wherein the stressed cells peaks in a flowcytometric histogram or a cytogram in the range of from 0% to 90% relative to the normal cells, such as 0-70%, e.g. 0-60, such as 0-50%, e.g. 10-40, such as 10-30%, e.g. 10-20.

15. A method according to claim 1, wherein the stressed cells peaks in a flowcytometric histogram or a cytogram in the range of from 105% and above relative to the normal cells, such as 110% and above, e.g. 120% and above, such as 130% and above, e.g. 140% and above, such as 150% and above, e.g. 200% and above.

16. A method according to claim 1, wherein the determination of one or more virus infected cell(s) is/are independent on the type of virus and/or whether one or more type(s) of virus is/are infecting.

17. A method according to claim 1, wherein the virus is a bacteriophage, such as a bacteriophage that infect Gram positive bacteria, such as a bacteriophage that infect lactic acid bacteria, such as a bacteriophage that infect lactococcus species, such as a bacteriophage that infect lactococcus species selected from the group consisting of type 936, type P335 and type C2.

18. A method according to claim 1, wherein a computer is used for observing the change in the sample caused by the virus infected cell(s).

19. A method according to claim 18, wherein the computer is provided with a suitable computer program for detecting the change.
